(19) United States
(12) Patent Application Publication
(10) Pub. No.: US 2005/0203327 A1
(43) Pub. Date: Sep. 15, 2005

(54) Hydrocarbon separation process

(76) Inventors: Stevan Jovanovic, North Plainfield, NJ (US); Kirk Walton Limbach, Dresher, PA (US); Ravi Jain, Bridgewater, NJ (US); Frank R. Fitch, Bedminster, NJ (US); Martin Bulow, Watchung, NJ (US); Seungdoo Park, Watchung, NJ (US)

Correspondence Address: Philip H. Von Neida;The BOC Group, Inc., Legal Services-IP, 575 Mountain Ave., Murray Hill, NJ, 07974, US

(21) Appl. No.: 11/070,466

(22) Filed: Mar. 2, 2005

Related U.S. Application Data
(60) Provisional application No. 60/551,583, filed on Mar. 9, 2004.

(51) Int. Cl. C07C 7/12; B01D 53/02

(52) U.S. Cl. 585/820; 585/826; 95/143

(57) ABSTRACT

The present invention provides for a process of separating hydrocarbons such as short chain paraffins and olefins from non-hydrocarbon gases using short-cycle time concentration swing adsorption processes. The hydrocarbons are adsorbed from the gaseous stream on highly siliceous nanoporous materials, e.g., such as of aluminum-deficient faujasite-type zeolites, by way of a pressure, vacuum or temperature swing adsorption, then desorbed from the adsorbent in the presence of steam. Optionally, the steam is desorbed from the adsorbent through the use of air or inert gas or a recycle of the waste gas effluent. The invention also provides for a new method of preparing novel shapes such as beads and monolithic structures of the highly sileceous nanoporous materials.

The present invention claims priority from U.S. Provisional Patent Application Ser. No. 60/551,583, filed Mar. 9, 2004.

### BACKGROUND OF THE INVENTION

The present invention provides for a hydrocarbon separation process from non-hydrocarbon gases such as nitrogen, carbon monoxide and carbon dioxide. Hydrocarbons separated by such a process include but are not limited to short chain (C₁ to C₅) paraffins and olefins (for example ethane, ethylene, propane, propylene, butanes, and butylenes).

Certain petrochemicals are produced commercially by the partial oxidation of an appropriate hydrocarbon in the vapor phase over a suitable catalyst and in the presence of an oxygen-containing gas. For example, cyclic anhydrides are produced commercially by the vapor phase catalytic partial oxidation of aromatic hydrocarbons, such as o-xylene or benzene, or straight-chain hydrocarbons, such as n-butane, or butene, in the presence of an oxygen-containing gas, over a vanadium-containing catalyst. Similarly, nitrites, alkylene oxides, aldehydes and halogenated hydrocarbons are produced by the partial oxidation of appropriate alkanes and alkenes in the presence of selected catalysts.

Air is generally used as the oxygen-containing gas, because of its low cost and ready availability. Oxygen-enriched air is also used. The reaction can be carried out in any suitable reactor, such as a fixed bed, a fluidized bed, a moving bed, a trickle bed or a transport bed reactor, and it produces the petrochemical, and generally carbon monoxide (CO), carbon dioxide (CO₂), water, and smaller amounts of other partially oxidized by-products. The reaction equipment train generally consists of a reactor, in which the petrochemical product is produced, a scrubber, in which the petrochemical product is scrubbed from the reactor effluent gases by means of water or other solvent for the petrochemical, and means for further treating the scrubbed effluent gases.

Currently, it is common to practice the above-described process on a single pass basis with the conversion of hydrocarbon to the desired petrochemical product being maximized. This results in a low overall efficiency, since the selectivity to petrochemical product is below the maximum. Consequently, the scrubber effluent gas contains considerable amounts of CO and CO₂, in addition to unreacted hydrocarbon. These products are usually incinerated, so that the only return realized from them is heat value. In modified processes, a portion of the scrubber effluent gas is recycled, the conversion of the hydrocarbon feedstock is lowered and the selectivity of hydrocarbon conversion to the desired petrochemical product is increased. The remainder of the effluent are purged from the system to prevent the build-up of CO, CO₂ and nitrogen (introduced into the system when air is used as the source of oxygen). These improvements results in a reduced “per pass” conversion, but the overall efficiency of the process is increased.

Typical processes do not make allowance for moisture contained in the gaseous effluent from the partial oxidation product recovery unit and in purge air, when ambient air is used to purge the adsorbent that is employed to separate hydrocarbons from the waste gas stream. Moisture is produced in the partial oxidation reaction; accordingly, the hot gaseous effluent from the reactor contains moisture. As the effluent gas passes through the product scrubber some moisture may be removed by condensation due to cooling of the gas stream, if an aqueous solvent is used. When a nonaqueous solvent is used moisture is not permitted to condense. In any event, the gas stream leaving the scrubber still contains moisture, and in fact can be saturated with moisture, even if a nonaqueous scrubbing agent is used. Moisture is more strongly adsorbed than the unreacted hydrocarbons and carbon oxides by conventional adsorbents; accordingly, unless the moisture is removed from the gas stream entering the adsorption units, it will be preferentially adsorbed onto the adsorbent, thereby reducing the capacity of the adsorbent for hydrocarbon adsorption.

The problem of moisture is further aggravated when ambient air is used as a purge gas for regeneration of the beds of adsorbent. Ambient air contains moisture; thus, moisture will replace the hydrocarbon being desorbed from the adsorption beds during the purge step when the beds are purged with the air. This will further reduce the capacity of the adsorbent during the adsorption step of the following cycle.

It is known to remove moisture from ambient air or a gas stream by various techniques. For example the air and gas streams can be dried by passing the air and gas stream through desiccants.

The present invention would allow such reactions to be run with higher selectivity although with a lower per pass conversion, which in combination with the proposed efficient recovery and recycle of unreacted hydrocarbons, increases the overall yield. Since the products would be recycled, pollution and costs associated with the incineration of unreacted hydrocarbons should be significantly reduced.

### SUMMARY OF THE INVENTION

The present invention provides for a process of separating a hydrocarbon from non-hydrocarbon gases comprising two steps in a short-cycle time concentration swing adsorption system. In the first step, the hydrocarbon is adsorbed on a highly siliceous nanoporous materials, e.g., such as of aluminum-deficient faujasite-type zeolites (for instance USY or DAY) in order to increase stability in the presence of water and/or steam and the hydrophobicity of the material. The preparation of such materials with these advantageous properties by shaping into monolithic structures but also beads and other shapes comprises an aspect of the invention. The hydrocarbon is then desorbed in the next step using steam, which may then be condensed out of the resulting effluent to recover the hydrocarbon for further use.

In a third optional step, the steam is desorbed from the adsorbent through the use of air, or inert gas (such as nitrogen), or recycle of the lean gas (waste) effluent which is produced in the first step. The process is typically conducted at approximately 100° C. to 200° C. and a pressure of 1 to 2 bar.

A major advantage of this process is that a residual loading of process water during the entire series of cycles is possible, which enables utilization of the material in hot and wet process surroundings without losing either its chemical identity, nanoporosity or even advantageous hydrocarbon adsorption properties. This only becomes possible due to a specific type of pretreatment of the basic materials as described later before any shaping process takes place.

The new concept for hydrocarbon separation from inerts makes use of steam to regenerate the adsorbent in a very short cycle time, thus providing the following advantages:
(1) significantly smaller size and capital cost
(2) significantly reduced operating expense
(3) complete elimination of vacuum requirement
(4) elimination of flammability issues
(5) higher purity/quality product.

These advantages are realized due to the novel way of pretreating and shaping the basic highly siliceous nanoporous materials of this invention.

The major benefits of this process as compared to earlier processes for hydrocarbon separation are that no vacuum system is required, thus lowering capital and operating expense and increasing the intrinsic safety. The system is much smaller than previous pressure swing adsorption (PSA) or vacuum swing adsorption (VSA) systems due to the short cycle time which results in lowering capital expenditure and footprint size as well as operating expenses. A higher quality of hydrocarbon concentration of product due to a lower concentration of inerts in the hydrocarbon product stream will also result. Process operation under the conditions of utilizing super-heated steam, which is the main advantage of this invention, becomes possible as a result of the material pretreatment mentioned above and to be described further.

For processes which operate under vacuum, the risk of air ingression exists. The preferred pressure range for the proposed invention is close to or slightly over atmospheric pressure thus minimizing these risks. Furthermore, flammability issues are typically an important concern when PSA, VSA or temperature swing adsorption (TSA) processes are used to recover hydrocarbons from oxygen containing mixtures or when the adsorbent regeneration step utilizes air or other oxygen-containing gases. In the present invention, the use of steam to regenerate the adsorbent minimizes this concern.

In addition to the above mentioned advantages, the present invention also produces higher quality hydrocarbon product which has a higher concentration of hydrocarbon and less non-condensable gas inerts than would be obtained in classical PSA, VSA or TSA processes. This is accomplished through the use of steam for adsorbent regeneration as opposed to the use of air or other non-condensable gases. Nanoporous highly siliceous materials, for example, dealuminated zeolites (DAY) and ultra-stable zeolites (USY) are not particularly sensitive to water or steam contact as are some other adsorbents. These materials are not limited in their manufacture, particularly of specific shapes such as monolithic structures and beads, when made by the pretreatment and shaping processes as described by the present invention.

The adsorbent in this process may be in the form of pellets or in a structured packing or other suitable form. Structured packing would typically have the advantage of allowing a higher linear velocity of gases passing through the adsorbent bed. High linear velocities are desirable to allow for short cycle times on the order of but not limited to about 0.001 to about 600 seconds, preferably about 0.1 to about 60 seconds, more preferably from about 3 to about 8 seconds per step.

The pressure of the proposed process could vary from about 0.1 to about 20 bar with a range of about 0.3 to about 3 bar preferred.

Considering the preferred pressure range which is slightly above atmospheric, the corresponding bed temperature for the proposed process should be above 100° C. in order to allow steam to be effectively used as a purge agent. However, the temperature of the process could vary from about 40° C. to about 300° C., preferably from about 100° C. to about 200° C.

Superheated steam may be used to desorb hydrocarbon and prevent steam condensation in the bed. Preferably, effective heat recovery of process steam is achieved in the process. Therefore, efficient heat recycle through the use of heat exchangers to recover heat energy is desirable. In one embodiment of the invention, the product mixture, which includes desorbed hydrocarbon and steam, transfers at least some heat to the stream of hot water, which is condensed water from the product mixture, through a heat exchanger. Hot water becomes preheated steam, and then the quality of this stream of steam is raised by adding heat through an additional heat exchanger. As a result, superheated steam is prepared for the next cycle.

An additional benefit of the present invention is that the lean gas, which is composed of mainly inert gases, can be recycled in order to enhance heat recovery. Depending on the compositions of waste stream or demands of product quality, hot air or inert gas may be used to desorb steam in a separate step. Therefore, additional energy to increase the temperature of air or inert gas may be desirable in that case and may allow for improved and more efficient hydrocarbon recovery.

The lean gas produced during the adsorption step may be transferred to a buffer tank to be available for any disturbance in the process. The amount of lean gas to recycle to the bed may be determined by that needed to desorb the rest of the steam in the bed to a desired level.

A main advantage of this invention is the usage of superheated steam conditions. This advantage is largely caused by the presence of a certain mesoporosity within the primary adsorbent particles due to the acid and thermal treatment of this invention, before the shaping procedure takes place. Within these mesopores, but not within the secondary porosity of beads, etc., process water remains trapped even during the desorption step. Thus, no reduction occurs in both the adsorption capacity and selectivity of the adsorbent material towards hydrocarbons.

In another embodiment of the present invention, a method is provided wherein air or inert gas is introduced across the bed instead of lean gas. Fresh air or nitrogen can be more effective to purge than recycled lean gas for some cases; however, in the case of air, flammability is a concern and should be taken into consideration in order to practice the invention and perform the process safely. The air or inert gas would preferably be heated before introduction into the bed to maintain a desired temperature and desorb the majority of steam in the bed.

In another embodiment of the present invention, the stream of lean gas is combined with air or inert gas through a mixing device to form a purge stream. This allows regulation of the amount of oxygen content fed to the bed and maintains a desired flammability range. The air or inert gas preferably is heated before mixing with lean gas to maintain a desired temperature.

### BRIEF DESCRIPTION OF THE DRAWINGS

FIG. 1 is a schematic representation of a short cycle time hydrocarbon separation process.

FIG. 2 is a schematic representation of a short cycle time hydrocarbon separation process using a different stream to desorb steam in the adsorbent bed.

FIG. 3 is a schematic representation of a short cycle time hydrocarbon separation process with a modification of the lean gas stream to desorb the steam in the adsorbent bed.

### DETAILED DESCRIPTION OF THE INVENTION

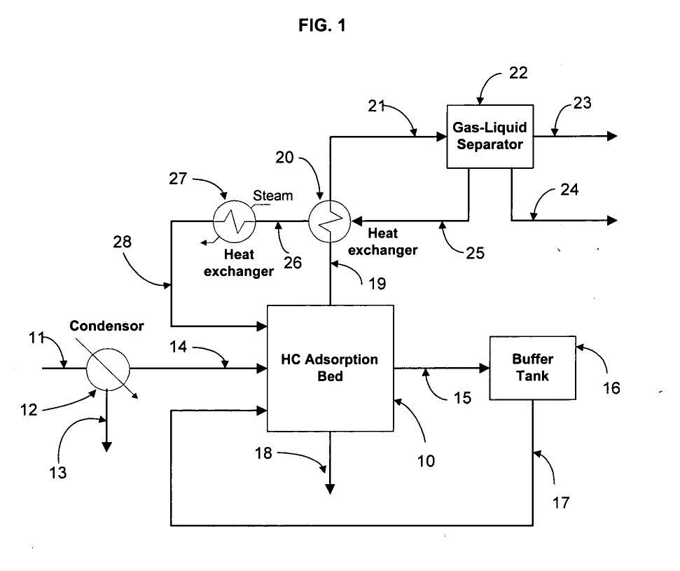

FIG. 1 describes one embodiment of the invention for a short-cycle-time hydrocarbon separation process including partial heat recovery. Waste feed stream 11 is fed to condenser 12 to reduce water content of feed stream 11, and then introduced to the hydrocarbon adsorption bed 10. The hydrocarbons in the waste stream are adsorbed on the adsorbent (zeolite Y in this example) which has a high selectivity for hydrocarbons. During the adsorption, the hot lean gas 15 comprising a majority of inert gas and a small amount of water is fed to buffer tank 16.

After the adsorption step is completed, superheated steam is discharged to hydrocarbon adsorption bed 10 to desorb the hydrocarbon on the adsorbent. The process of desorption is completed in a short time. The product mixture leaving the hydrocarbon adsorption bed is composed of hydrocarbon with superheated steam including a small amount of inerts. The stream of product mixture is fed to heat exchanger 20 through line 19. At the heat exchanger 20, heat of the product mixture is transferred to a stream of hot water 25, which is from a gas-liquid separator 22. After the product mixture loses heat energy through the heat exchanger, it is fed to gas-liquid separator 22 through product mixture line 21. Hydrocarbon as a final product and condensed water are separated in a gas-liquid separator.

The required condensed water for the next cycle is recycled through the line 25, and excess water is separated off as stream 24. Therefore, a part of the water containing dissolved CO₂ in water is purged off continuously. The preheated steam 26 which is formed through heat exchanger 20 is fed to additional heat exchanger 27 to produce superheated steam 28 and ready to be introduced to hydrocarbon adsorption bed 10 for next cycle. Before the bed is mixed with waste feed stream 14 in the next cycle, the bulk of the steam in the bed is desorbed to lean gas purge line 18 by recycled lean gas stream 17 from the buffer tank 16.

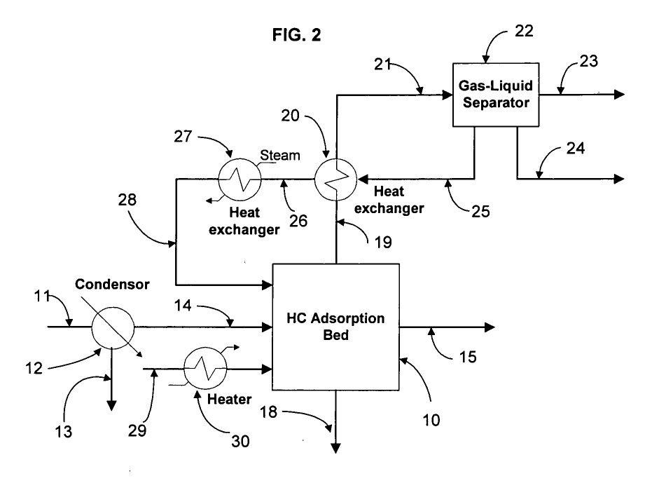

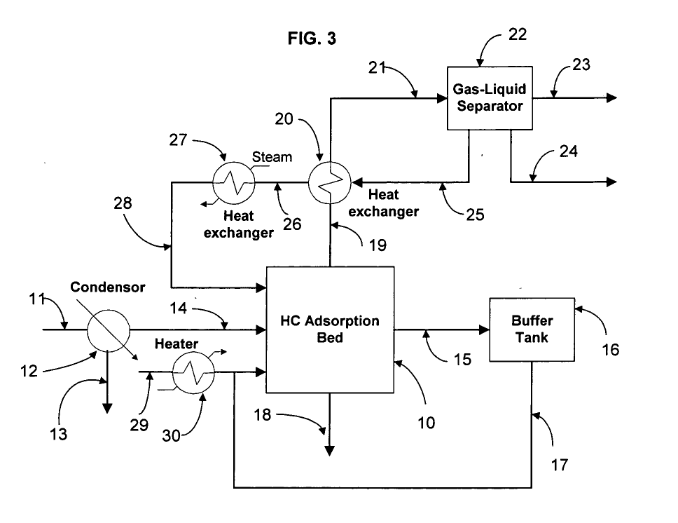

For purposes of FIGS. 2 and 3, all the numbers present in FIG. 1 and their accompanying description are the same in FIGS. 2 and 3.

As shown in FIG. 2, another embodiment of the separation system makes use of different stream to desorb the bulk of the steam in the bed. A stream of ambient air or inert gas 29 is fed to gas heater 30 to maintain a desired temperature to desorb the majority of the steam in the bed. If hot process air or inert gas is available nearby, then a gas heater may not be necessary. The hot lean gas 15 is purged from the separation system without recycling. In the case of air, the introduction of the waste feed stream should be controlled carefully due to the flammability of hydrocarbon in air.

FIG. 3 shows an embodiment with modification of the lean gas stream to desorb the majority of the steam in the bed. The recycled lean gas stream 17 is mixed with a stream of ambient air or inert gas 29 which is heated by heater 30 to maintain a desired temperature. If hot process air or inert gas is available nearby, then the gas heater may not be necessary. The combination of lean gas and air or inert gas may allow for safer operation than air alone.

This application further relates to the utilization of highly siliceous micro- and meso porous (i.e., nanoporous) adsorbent materials such as molecular sieve/zeolite type materials in specific shapes such as monoliths and beads. These materials are made based on novel methods for making monoliths and beads. For example, beads are superior to other shapes such as cylinders and hollow cylinders is due to their advantages in pressure drop behavior of packed adsorber columns and mechanical stability under the influence of frequently changing pressures in both pressure swing adsorption (PSA) and temperature swing adsorption (TSA) processes. Additionally, this superior performance is shown by significantly increased gas flow rates, significantly accelerated mass transfer due to an optimum in surface-to-volume ratio and regulation of macrokinetics within the pore system of beads, and maximized packing density of the adsorbent particles in adsorber columns.

For these reasons, it has been preferred in adsorption processes to use beads rather than cylinders or even hollow cylinders. There is also an additional advantage in utilizing even more compact secondary adsorbent structures such as monoliths that allow the specific and significant reduction in the cycle time in processes of their practical utilization. Neither beads nor monolithic shapes were available/accessible for highly siliceous micro- and mesoporous (nanoporous) adsorbent and catalyst materials, and specifically for such materials as, for example, of DAY or USY-type zeolites.

In addition, these binders, viz., those with a value pH >10 in their water suspensions, can be utilized for the manufacture of other sorbent shapes, beside those of monoliths, beads, extrudates, solid and hollow cylinders, beads and cylinders with non-porous inner cores, etc., due to a specific material pretreatment. Indeed, not following that procedure, will result in the basic microcrystalline nanoporous adsorbent material being destroyed during the shaping process.

Further, there is no restriction with regard to the primary crystal size range or with regard to the upper limit of binder content which could be as high as 85 weight percent.

This invention further relates to the manufacture of monolithic structures and beads of a series of pulverulent crystalline nanoporous materials such as of the zeolites types of aluminum-deficient Y (faujasite) type such as of its sub-types DAY (dealuminated Y) and USY (ultrastable Y), furtheron, Beta, erionite, mordenite, silicalite-1, silicalite-2, Theta-1, Theta-3, ZSM-3, ZSM-5, ZSM-11, ZSM-12, ZSM-20, and their mixtures, and mesoporous materials MCM-41 and MCM-48, and mixtures thereof. The manufacturing technique is applicable to many different materials, the manufacture of monolithic shapes and beads before being of great difficulty.

The present invention provides for the use of modern and highly productive beading principles and related techniques such as those of so-called Eirich mixers and rotary table granulators, which ensure homogeneity in bead size in narrow factions of a broad general range, i.e., (0.5 to 8 mm), in conjunction with aiming for and guaranteeing homogeneity in bulk density, and macro- and mesoporosity, and, hence, mass transfer properties. These techniques can be automated.

It is very difficult to achieve such parameter values by extrusion and kneading-screw techniques described in patent literature, cf., Sextl et al. (U.S. Pat. No. 5,316,993). For example, the bulk density achievable by extrusion of DAY zeolite as described therein falls in a range of about (0.4-0.5) g/cm³, which is known to those skilled in this art. Such a low bulk density is connected with low mechanical stability. In contrast to this result, beading of DAY zeolite in accordance with the present invention allows it to achieve bulk densities that amount to (0.6-0.75) g/cm³, which, in addition, can be controlled in narrow factions. If all other parameters were kept constant, such a difference in bulk density alone allows for minimizing adsorber and/or reaction vessels by about 25 to 30% resulting in significant cost savings.

The shaping techniques and procedures for beading for highly siliceous nanoporous materials are based on entirely unexpected findings with regard to the use of binders (pH value) and basic nanoporous materials (pretreatment), the combination of these factors allows the development of a new method for shaping those materials into beads.

The invention further relates to an additional stabilization step with the following features:

Prior to its mixing with a binder and subsequent shaping, the pulverulent crystalline nanoporous material undergoes a heat treatment step, at a temperature between 600 and 1000° C. This additional stabilization of the dry material before its shaping step should be executed for a duration that is specific with regard to its particular nature. This heat treatment prior to contacting the crystalline material with binder must be distinguished from the heat treatment after shaping, which is the final activation/calcination step for setting the binder system, and which may take place at temperatures within the same range, for highly siliceous nanoporous materials.

If the pulverulent crystalline nanoporous material belongs to the group of ultra-stable Y-type zeolites, which as a rule are obtained by (water) steam dealumination, the material must undergo an acid treatment prior to the heat treatment step of this invention, i.e., prior to the shaping procedure. This acid treatment, e.g., by hydrochloric acid at a pH value of about 1 to 1.5 at ambient temperature may proceed multiply, before the filter cake dried undergoes the heat treatment step of this invention, i.e., prior to the shaping procedure.

After these stabilization procedures any type of the known binders can be utilized for shaping of the highly siliceous pulverulent crystalline nanoporous materials, and no restriction exists anymore with regard to the pH value of their slurries with water, whether it amounts to a value pH <10 or pH >10. This unexpected feature in turn allows for the utilization of modern and highly productive beading principles and related techniques such as those of so-called Eirich mixers and rotary table granulators, which ensure manufacture of beads with the advantages listed above.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

1. A method for separating a hydrocarbon gas from a mixture of gases comprising the steps:
a) passing said gas mixture through an adsorbent bed containing a highly siliceous nanoporous adsorbent, wherein said hydrocarbon is adsorbed by said highly siliceous nanoporous adsorbent; and
b) passing steam through said adsorbent bed thereby desorbing said hydrocarbon; and
c) recovering a product stream comprising said hydrocarbon.

2. The method as claimed in claim 1 wherein said highly siliceous nanoporous adsorbent is an aluminum-deficient faujasite-type zeolite.

3. The method as claimed in claim 2 wherein said aluminum-deficient faujasite-type zeolite is selected from the group consisting of dealuminated Y-type zeolite, ultrastable Y-type zeolite, furtheron, Beta, erionite, mordenite, silicalite-1, silicalite-2, Theta-1, Theta-3, ZSM-3, ZSM-5, ZSM-11, ZSM-12, ZSM-20, and mixtures thereof, and MCM-41 and MCM-48 and mixtures thereof.

4. The method as claimed in claim 2 wherein said aluminum-deficient faujasite-type zeolite is dealuminated Y-type zeolite.

5. The method as claimed in claim 1 wherein said highly siliceous nanoporous adsorbent is in the shape of a monolith.

6. The method as claimed in claim 1 wherein said highly siliceous nanoporous adsorbent is in the shape of a bead.

7. The method as claimed in claim 1 wherein said product stream further contains steam.

8. The method as claimed in claim 7 wherein heat is recovered from said product stream.

9. The method as claimed in claim 8 wherein said heat is recovered with a heat exchanger.

10. The method as claimed in claim 1 wherein the pressure of said gas mixture is about 0.1 to about 20 bar.

11. The method as claimed in claim 1 wherein said pressure is about 0.3 to about 3 bar.

12. The method as claimed in claim 1 wherein the temperature of said gas mixture is about 40° to about 300° C.

13. The method as claimed in claim 12 wherein said temperature is about 100° to about 200° C.

14. The method as claimed in claim 1 said steam is superheated steam.

15. The method as claimed in claim 1 wherein said hydrocarbon is selected from the group consisting of short chain paraffins and olefins.

16. The method as claimed in claim 1 further comprising the step of desorbing said steam from said adsorbent by passing a gas stream over said adsorbent.

17. The method as claimed in claim 15 wherein said gas stream is selected from the group consisting of air, recycled lean gas, an inert gas, and waste gas.

18. The method as claimed in claim 17 wherein said gas stream is recycled lean gas.

19. A method of separating a hydrocarbon gas from a mixture of gases in a cyclical process comprising the steps:
(a) passing said mixture of gases through an adsorbent bed, wherein said adsorbent bed contains a highly siliceous nanoporous adsorbent and said hydrocarbon gas is adsorbed by said adsorbent;
(b) passing steam through said adsorbent bed thereby desorbing said hydrocarbon gas; and
(c) recovering a product stream comprising said hydrocarbon gas.

20. The method as claimed in claim 19 wherein said cyclical separation is selected from the group consisting of concentration swing adsorption, pressure swing adsorption, vacuum swing adsorption, temperature swing adsorption, and combinations thereof.

21. The method as claimed in claim 19 wherein said highly siliceous nanoporous adsorbent is an aluminum-deficient faujasite-type zeolite.

22. The method as claimed in claim 21 wherein said aluminum-deficient faujasite-type zeolite is selected from the group consisting of dealuminated Y-type zeolite, ultrastable Y-type zeolite, furtheron, Beta, erionite, mordenite, silicalite-1, silicalite-2, Theta-1, Theta-3, ZSM-3, ZSM-5, ZSM-11, ZSM-12, ZSM-20, and mixtures thereof, and MCM-41 and MCM-48 and mixtures thereof.

23. The method as claimed in claim 22 wherein said aluminum-deficient faujasite-type zeolite is dealuminated Y-type zeolite.

24. The method as claimed in claim 19 wherein said aluminum-deficient faujasite-type zeolite is dealuminated Y-type zeolite.

25. The method as claimed in claim 19 wherein said highly siliceous nanoporous adsorbent is in the shape of a monolith.

26. The method as claimed in claim 19 wherein said highly siliceous nanoporous adsorbent is in the shape of a bead.

27. The method as claimed in claim 19 wherein said product stream further contains stream.

28. The method as claimed in claim 26 wherein heat is recovered from said product steam.

29. The method as claimed in claim 27 wherein said heat is recovered with a heat exchanger.

30. The method as claimed in claim 19 wherein the pressure of said gas mixture is about 0.1 to about 20 bar.

31. The method as claimed in claim 19 wherein said gas pressure is about 0.3 to about 3 bar.

32. The method as claimed in claim 19 wherein the temperature of said gas mixture is about 40° to about 300° C.

33. The method as claimed in claim 19 wherein said temperature is about 100° to about 200° C.

34. The method as claimed in claim 19 wherein said cycle ranges from about 0.001 to about 600 seconds per step.

35. The method as claimed in claim 19 wherein said cycle ranges from about 0.1 to about 60 seconds per step.

36. The method as claimed in claim 19 wherein said cycle ranges from about 3 to about 8 seconds per step.

37. The method as claimed in claim 19 said steam is superheated steam.

38. The method as claimed in claim 19 wherein said hydrocarbon is selected from the group consisting of short chain paraffins and olefins.

39. The method as claimed in claim 19 further comprising the step of desorbing said steam from said adsorbent by passing a gas stream over said adsorbent.

40. The method as claimed in claim 39 wherein said gas stream is selected from the group consisting of air, an inert gas, and waste gas.

41. The method as claimed in claim 40 wherein said gas stream is recycled lean gas.

42. A method for preparing a highly siliceous nanoporous adsorbent comprising the steps:
a) heating a crystalline nanoporous material;
b) mixing said heated crystalline nanoporous material with a binder; and
c) shaping said mixture.

43. The method as claimed in claim 42 wherein said highly siliceous nanoporous adsorbent is an aluminum-deficient faujasite-type zeolite.

44. The method as claimed in claim 43 wherein said aluminum-deficient faujasite-type zeolite is selected from the group consisting of dealuminated Y-type zeolite, ultrastable Y-type zeolite, furtheron, Beta, erionite, mordenite, silicalite-1, silicalite-2, Theta-1, Theta-3, ZSM-3, ZSM-5, ZSM-11, ZSM-12, ZSM-20, and mixtures thereof, and MCM-41 and MCM-48 and mixtures thereof.

45. The method as claimed in claim 44 wherein said aluminum-deficient faujasite-type zeolite is dealuminated Y-type zeolite.

46. The method as claimed in claim 42 wherein said binder has a pH value greater than 10 in their water suspensions.

47. The method as claimed in claim 42 wherein said crystalline nanoporous material is heated at a temperature between 600 and 1000° C.

48. The method as claimed in claim 42 wherein said heating may be preceded by an acid treatment of said crystalline nanoporous material.

49. The method as claimed in claim 42 wherein said shaping is performed by mixers and rotary table granulators.
